(19) United States
(12) Patent Application Publication
(10) Pub. No.: US 2021/0369908 A1
(43) Pub. Date: Dec. 2, 2021

(54) METHOD FOR DUAL MODE DISINFECTION IN A VEHICLE INTERIOR

(71) Applicant: GHSP, Inc., Holland, MI (US)

(72) Inventors: Aghuinyue E. Umenei, Caledonia, MI (US); Ian P. Sage, West Olive, MI (US); Richard W. Harris, Wyoming, MI (US); Josiah W. Bonewell, Grand Rapids, MI (US); Samuel N. Gustafson, Holland, MI (US)

(73) Assignee: GHSP, Inc.

(21) Appl. No.: 17/398,732

(22) Filed: Aug. 10, 2021

Related U.S. Application Data
(63) Continuation-in-part of application No. 16/736,691, filed on Jan. 7, 2020, now Pat. No. 11,083,810.

(51) Int. Cl. A61L 9/20; A61L 2/10

(52) U.S. Cl. CPC A61L 9/20; A61L 2/10; A61L 2209/12; A61L 2202/25; A61L 2209/15; A61L 2202/11; A61L 2202/14; A61L 2209/111

(57) ABSTRACT

A method for providing dual mode UV automotive disinfection includes the step of providing one or more UV light sources and moving automotive cabin air in proximity to the light sources. A shutter is configured in proximity to the light source in either an open or closed position where operation of the light source(s), motor and shutter are controlled where the light sources operate in a first mode by disinfecting cabin air in proximity to the light source or in a second mode using the at least one light source to irradiate cabin surfaces.

### FIELD OF THE INVENTION

The present invention relates generally air and surface disinfection and more particularly to a system and methods for providing dual mode disinfection in vehicles.

### BACKGROUND

Applying a safe and useful dose of Ultra-Violet C (UV-C) light for disinfection within a vehicular environment presents several problems. These include the need to maintain the safety of an operator, or anyone in the vicinity of the UV-C. UV-C light in unsafe doses can present a hazard to the user and consequently excessive exposure must be avoided.

Disinfection often requires providing an effective dose of UV-C radiation to multiple surfaces within the vehicle. If too much UV-C light is applied to certain materials, it can cause damage to the material. The optimization of disinfection exposures and dosing configurations are preferred.

Thus, solutions are needed to safely and efficiently disinfect both air and surfaces in a vehicle that are in frequent contact with its occupants.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 is a perspective view of a control panel used in a human machine interface (HMI) in accordance with an embodiment of the invention.

FIG. 2 is a side cross-sectional view of the control panel shown in FIG. 1.

FIG. 3A is side view illustrating a UV-C light source and dynamic multi-reflector assembly in accordance with the invention.

FIG. 3B is a perspective view of the dynamic multi-reflector assembly in operation.

FIG. 4 is a block diagram illustrating the operation of an HMI surface disinfection device in accordance with the invention.

FIG. 5A and FIG. 5B are graphs showing representations of the effects of distributing UV light on a touchscreen surface using micro-reflective surface intensity distribution as described herein.

FIG. 6A illustrates the housing used for UV dual mode disinfection where air is moved through the housing according to an alternative embodiment of the invention.

FIG. 6B illustrates the housing shown in FIG. 6A in an alternative mode where UV-C irradiates the interior of a vehicle.

FIG. 7 is a block diagram illustrating operation of the dual mode disinfection system.

FIG. 8 is a flow chart diagram illustrating method of operation used in the dual mode disinfection system.

FIG. 9 is a flow chart diagram illustrating the autocalibration process for a dual mode disinfection system.

FIG. 10 is a chart illustrating a vehicle differentiation (VD) table used in the autocalibration process of FIG. 9.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a control panel having UV disinfection. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

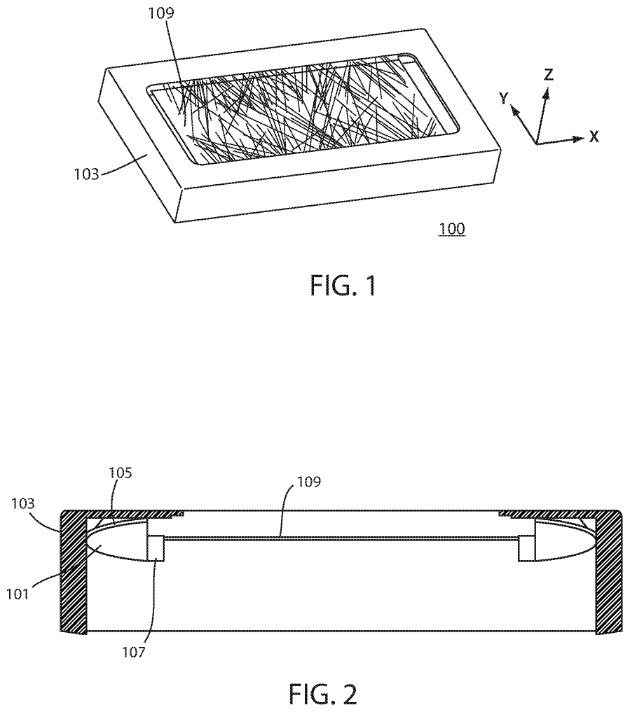

FIG. 1 is a perspective view of a control panel used in a human machine interface (HMI) in accordance with an embodiment of the invention. FIG. 2 is a side cross-sectional view of the control panel shown in FIG. 1. With regard to both FIG. 1 and FIG. 2, the HMI control panel 100 is shown as used with a liquid crystal display (LCD) touch screen or the like. Those skilled in the art will recognize that a control panel might be used with any electric device and can include but is not limited to any panel using touch screen technology or other type of software touch control such as a keyboard or the like.

The control panel 100 uses one or more UV-C light source(s) 101 mounted under one or more side or edges of a housing such as an overlay or bezel 103. Although UV-C light is used generically herein, those skilled in the art will recognize that any frequency of light in the UV spectrum may be used for disinfection and antimicrobial functions. The bezel material is typically constructed of a UV transmissive material such as quartz or perfluoro-alkoxy plastic (PFA). The overlay 103 may also include a coating on its rear-facing surface to enhance reflectively. Materials are important when using UV-C since most engineering materials absorb UV-C light instead of reflecting or transmitting it, and some materials degrade in the presence of UV-C. Those skilled art will recognize that the light sources 101 may be many different technologies including but not limited to light emitting diode (LED).

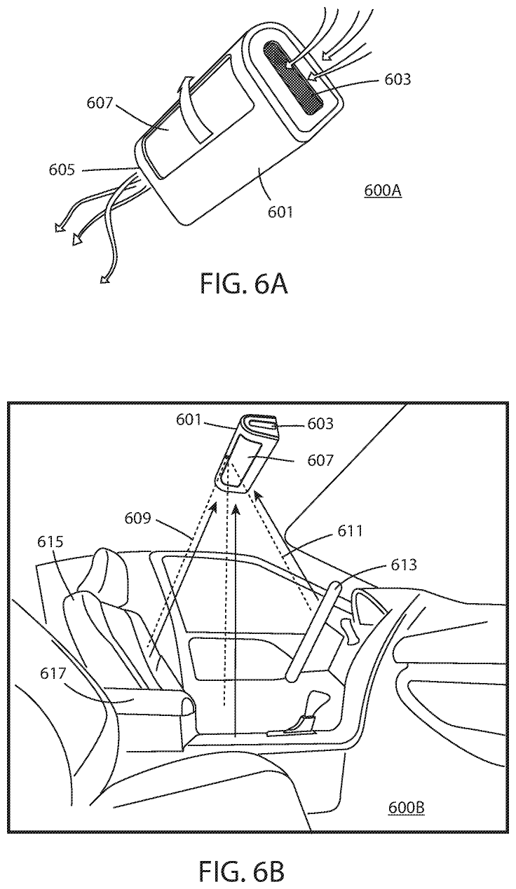

As will be described herein, a primary reflector 105 and secondary reflector 107 are both moveable and used to direct the LED's UV-C emission across the surface 109 of an HMI such as a touch screen LCD or the like. FIG. 1 illustrates the UV-C emission from one or more dynamically moveable multi-reflector assemblies that can extend the light in each of X, Y and Z planes across the surface 109 of the touch screen for any desired time interval. As described herein with regard to FIG. 6A and FIG. 6B and related embodiments herein, the reflector assemblies can also work like a door allowing light to escape rather than be reflected.

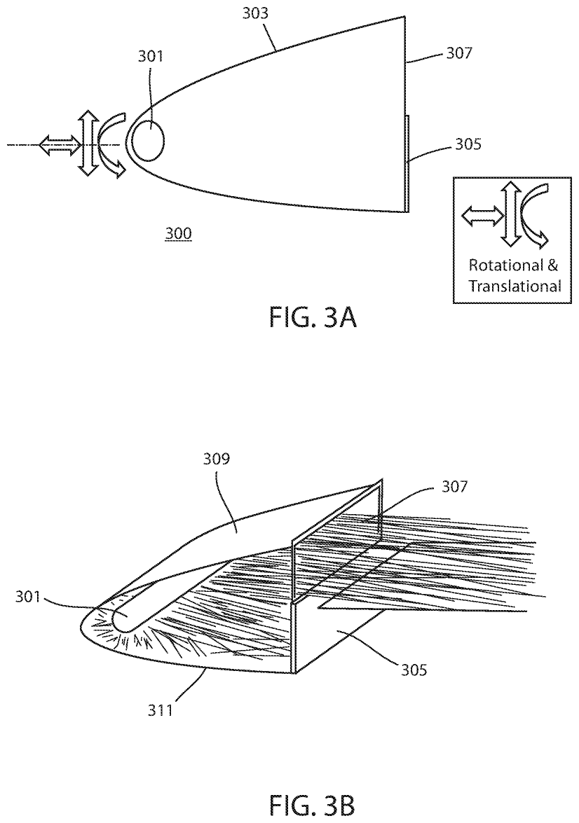

FIG. 3A is side view illustrating a UV-C light source and dynamic multi-reflector assembly in accordance with an embodiment of the invention. FIG. 3B is a perspective view of the dynamic multi-reflector assembly in operation. With regard to both FIG. 3A and FIG. 3B, the he dynamic multi-reflector assembly 300 include a UV light source 301 that is positioned at the back or rear of a primary reflector 303. The primary reflector 303 is substantially U-shaped so the UV light source is typically positioned at the center or back of the U configuration between an upper and lower reflector. In another embodiment, the primary reflector may be a parabola type shape.

A secondary reflector 305, is substantially flat in shape, and is positioned, forward and substantially orthogonally to the light source 301. In this embodiment, the secondary light source is at some predetermined distance from light source 301 and configured below its central axis of emission. The secondary reflector 305 is smaller in size than the opening of the primary reflector such that light escapes only from above the secondary reflector. This enables the secondary reflector 303 to reflect light toward the primary reflector 301 where the primary reflector 303 reflects light out of its front opening. A novel attribute of this design is that the reflector movement mechanism is both rotational and transactional. Thus, the primary reflector 301 and the secondary reflector 303 and be independently rotated and/or move to provide the desired amount or dose of light emission though a bezel aperture 307. The dynamic multi-reflector assembly 300 enables the dose of UV-C light to be precisely controlled for the optimum amount of pathogen disinfection. Thus, the dynamic multi-reflector assembly 300 offers dynamic movability since both the light source and reflectors can be moved to direct light emission to a specific location.

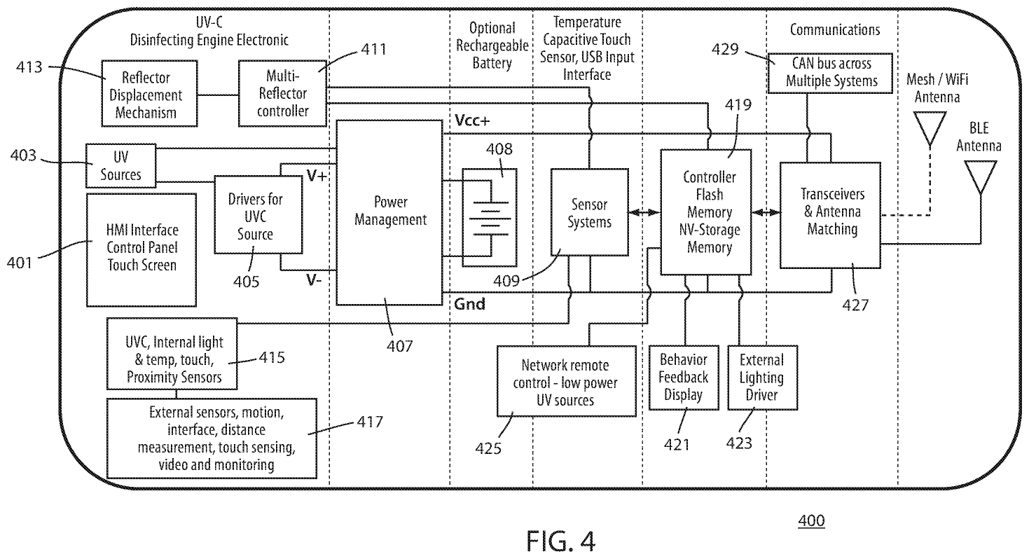

FIG. 4 is a block diagram illustrating the operation of an HMI surface disinfection device in accordance with the invention. FIG. 4 is a block diagram illustrating the operation of an HMI surface disinfection device in accordance with the invention. As seen in FIG. 4, the HMI control system 400 for controlling the HMI control panel 401 includes a plurality of UV sources 403 which are driven by one or more UV-C source drivers 405. Those skilled in the art will recognize that the UV light sources 403 may be cold cathode, low pressure Hg or UV-C LED's. The reactor is based flooding specific areas. As described herein, the UV lamp energy is variable and directed to programmed surfaces. The reflective surface control is programmed for variable intensity, distribution using a delivery mechanism. Mechanisms like micro-canti-level, linear motors or the like can be used to control direction and position of the light sources and reflectors. The UV light sources 403 are powered by a power management control 407 that is a power supply 408 that produces a regulated voltage from AC line voltage or a battery power source such as an optional battery. The battery may be size for the desired dose, interval and typical use cycle. Thus, the UV sources can also be used with a ballast or power source having power and UV-C feedback.

The power management control 407 also controls power to a sensor control 409. The sensor system 409 works to detect and control a multi-reflector controller 411 which operates a reflector displacement mechanism 413. As described herein, the reflector displacement mechanism works to control one or more reflectors used to direct the UV-C light to desired locations on the control panel 401—such as a touch screen. Further, the sensor system 409 also works with proximity sensors as well as internal light, temperature and touch sensors 415 to detect what areas of the touch screen are most often used. This allows the control system to determine desired areas of the touch screen where the UV-C light should be directed. Thus, the HMI control system 400 can actively target areas of a touch screen that are most likely or prone to harbor pathogens and/or other undesirable organisms from multiple user contact and high use. The UV sensor will confirm the type of dose and intensity information, and will track that dose over some predetermined time period. The sensor control 409 can also control a non-volatile storage memory 419 that stores all accumulated usage information and dosage data. Those skilled in the art will be also recognize that other types of external sensors can also be used to make this determination. These optional sensors can include but are not limited to motion, interface use, distance measurement, touch sensing and video sensors. Hence, the HMI using the UV delivery system as described herein uses dynamic moveable reflectors and can use direct feedback from HMI environment in order to track touches and events/cycles. As an example, the feedback sensors across the control panels/screens can include passive infra-red sensor (PIR) grids across the surface of the LCD screen with capacitive touch feedback from the screen function to determine use and UV disinfection requirements.

As seen in FIG. 4, the information stored in the memory 419 can be displayed 421 or used for controlling an external lighting driver 423. A remote network control 425 can also use this information for controlling additional low power UV sources 425. Wireless control using a Wi-Fi or Bluetooth is also accomplished using a transceiver and matching network 427. Antennas are optionally routed to outside ambient or external devices using on-board chip type antennas. This information can also be supplied to a controller area network (CAN) or local interconnect network (LIN) bus across multiple HMI control systems 429 using the transceiver 427 or the like. Hence, the dynamic move-ability of the multi-reflector assembly and sensor network monitoring and the HMI for positioning, as well as the known cycles of use etc. together make a more powerful system as compared to each individual system operating alone.

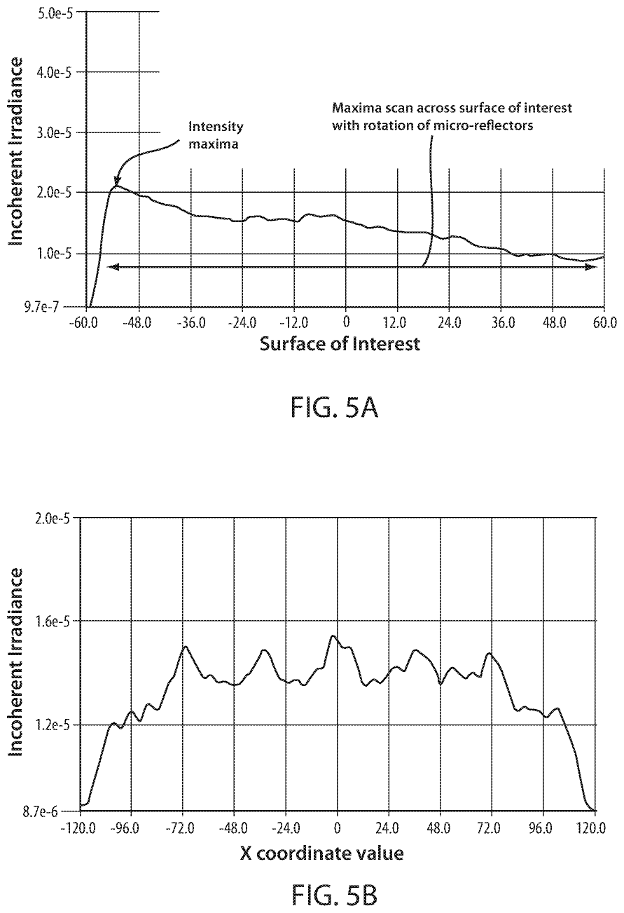

FIG. 5A and FIG. 5B are graphs illustrating the effect of micro-reflective surface intensity and its distribution effects. As seen in FIG. 5A, the intensity maxima 501 of the UV-C light can be controlled so it is greatest or maximized at any particular touchscreen location using the dynamic multi-reflector assembly. FIG. 5B illustrates the resulting scan distribution 503 where the dosage UV-C light is precisely controlled to ensure that the resulting distinction is uniform across the surface of the touchscreen.

In an alternative embodiment of the invention, FIGS. 6A and 6B illustrate a a device having a housing used in the system and method for dual mode disinfection as used in a vehicular interior. Dual mode disinfection means the disinfection of cabin air as well as the surfaces of various vehicle components. In this embodiment, these multiple disinfection modalities are combined in a single device. Those skilled in the art will also recognize, although this embodiment is directed to dual modality, embodiments with more than two modes are also possible.

Those skilled in the art will recognize the need to disinfect vehicle interiors, surfaces and air in enclosed spaces. However, providing UV-C light disinfection within such an enclosed space creates both a geometrical problem and a human interface problem. The geometrical problem concerns the disinfection area and light coverage within the vehicle. Prior art devices required the need for multiple devices or elements to disinfect both air and surfaces within these enclosed spaces. Various embodiments of the present invention provide solutions to this problem.

The human interface issue relates to the frequency of contact. When humans interact with vehicular spaces, a disinfection event and/or “cycle” is needed to UV cleanse a defined area. High touch locations such as steering wheel, seats, center console, touch screens, electronics switches, vehicle control interfaces and the like, are often contaminated with germs, bacteria and other pathogens because of their frequency hand contact. In order to disinfect the complete environment, these high touch areas need to be disinfected frequently. Detection related to the presence and/or absence of persons within this space is also required in order to know how and when to switch between air or surface treatments. Automating this process allows for frequent disinfection while maintaining a normal operating workflow between users or events.

FIG. 6A illustrates a device 600A used for UV-C dual mode disinfection where air is moved through the housing according to an alternative embodiment of the invention. A housing 601 is typically elongated in shape and may be configured with or attached to a vehicle headliner. A fan, using an electric motor inside the housing 600A, directs air longitudinally though the housing 601 from an air inlet 603. One or more UV-C lights within the housing 101, work to disinfect and cleanse the air when brought in proximity to the UV-C light. Thereafter, the cleansed and/or disinfected air is then directed through air outlet 605 where it can be distributed about the vehicle cabin. In this embodiment, a door or shutter 607 is shown in a closed position so to contain the UV-C light completely within the interior of the housing. In yet another embodiment, the UV-C lighting can be mounted on separate printed circuit boards allowing a first group of UV-C LEDs to be lit for air disinfection and a second groups of UV-C LEDs to be lit for surface disinfection. In this alternative embodiment, the use of a shutter may be optional.

FIG. 6B illustrates the device shown in FIG. 6A in an alternative or second mode showing a housing where UV-C light is irradiating the interior of a vehicle. FIG. 6B illustrates the dual mode nature of the system where the housing 601 is shown with the shutter 607 in an open position. This allows the UV-C light rays 609, 611 to contact various high contact interior surfaces of the vehicle including but not limited to the steering wheel 613, seat 615 and/or center console 617.

Those skilled in the art will further recognize that disinfection requires providing an effective dose of UV-C radiation to all surfaces of interest of the vehicle interior. Treating the air also requires an effective but different dose depending on a multiplicity of factors. Another alternative embodiment of the invention provides an algorithm and mechanical processes for determining the operating environment with a vehicle i.e. the amount of human contact. This allows the system to make a determination of when to disinfect and/or treat both the ambient air and interior vehicular surfaces with effective UV light dose with minimal device reconfiguration. To improve efficiency of air treatment within the same spatial confines of a surface treatment system, enhancements and modalities to the housing interior e.g. a UV cleaning chamber are designed into the device. These may include both aluminum (Al) reflectors on the shutter and a titanium dioxide (TiO₂) coating for photocatalytic oxidation. These features work to further enhance the disinfection process.

The human interface issue relates to the cycle of contact with vehicle interior surfaces. A human interacting with the interface is counted as an “event” or cycle that needs disinfection when completed. High touch events like touch screen use, vehicle actuation or ‘key” switches, vehicle control interfaces, vitals monitor that are used frequently are considered high touch devices. In order to disinfect the complete environment, these high touch areas in the vehicle cabin need to be frequently disinfected particularly those having inter-human usage. Automating this disinfection process allows for the normal operating workflow and between users or events in the vehicle. The need to disinfect surface and enclosed vehicular spaces creates problems in spatial geometry needed to disinfect ambient air as well as the interface with humans in this same space. Thus, a repeated reconfiguration of disinfection devices is often necessary depending on use case, vehicle type and pathogens involved.

In use, a geometrical problem exists with disinfection that relates to both space and light coverage. Embodiments used in the present invention are effective because spatial dimensions, irradiance and time are factored into the disinfection system configuration. These factors are all variable and depend upon different types and configuration of vehicles as well as the different types of pathogens present in the cabin space. Although various embodiments of the present invention can be manually configured to provide UV coverage, depending on the use case and the type of automobile, those skilled in the art will recognize that this manual disinfection process can be laborious and inefficient.

Hence, a further aspect of the dual mode disinfection system as described herein provides a system for the detection and registration of a series of events to trigger auto disinfection. The registration of a series of events is important to configuring the system to automatically automate the dual mode disinfection process. More specifically the control of the intensity of UV-C distribution enables enhanced efficacy, safety and versatility. By tracking movements and surfaces of interest distances with time-of-flight (TOF) sensors, unique configuration data can be used with methods or algorithms that consider different spatial implementations. This type of automated process provides for a greater ease of use of a vehicular disinfection system within the cabin interior and self-calibration becomes a part of the device's installation process.

Thus, by locating UV sensors in the farthest-reaching areas of the vehicle interior, UV-C intensity is detected and/or sensed allowing the UV-C light sources to be adjusted to a required dose. This spares the plastics and/or other materials used in the vehicle interior environment any additional UV-C exposure. Analytics can be generated using both internal and external exposure data the is captured to provide insight and algorithm upgrades. These processes and method provide value added data to customers using insight generated from analytic effects of the sensors and treatment data. These upgrades can take the form of a graded dosages due to workload or seasonal patterns detected in the data which can help users of the system improve efficiency of usage.

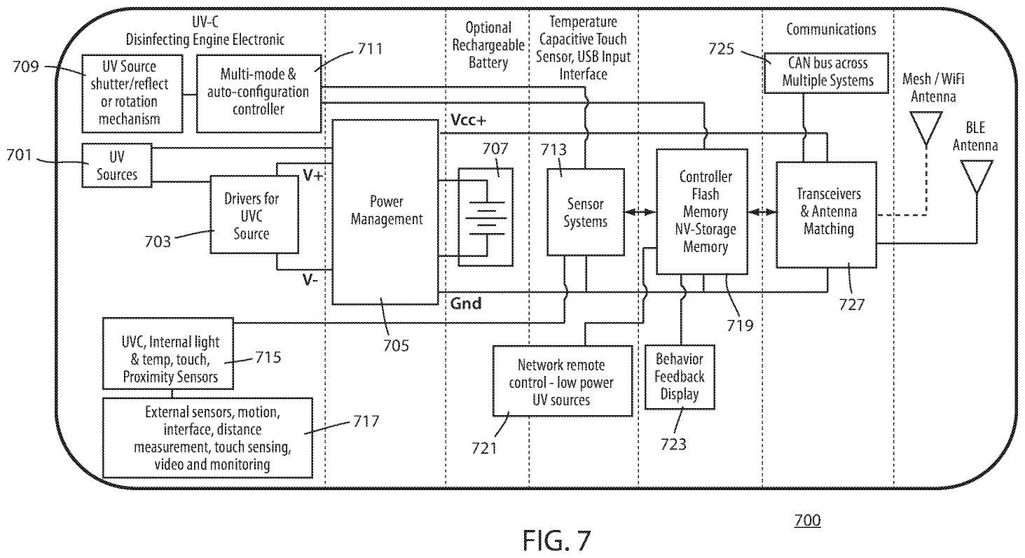

FIG. 7 is a block diagram illustrating the operating architecture of the dual mode disinfection system. Those skilled in the art will further recognize that the multimode disinfection system 700 is autoconfiguring and includes numerous components. One or more UV-C sources 701 utilize drivers 703 which are powered using a power management controller 705. A motor 702 operates to move cabin air though the housing when in a first or air disinfection mode. A battery backup 707 works back up the power management system in the event of power failure. As noted herein, the UV light sources 701, motor 702 and door or shutter 709 are controlled by a multimode auto-configuration controller 711. The controller 711 controls motor actuation and speed and also works to open or close the shutter depending on whether ambient air or vehicular interior surfaces are to be disinfected.

One or more sensors 713 work with the multimode controller 711 to determine spatial dimensions, irradiance and time into the control of the UV sources 701. Given an automotive vehicle often includes an in-built Local Interconnect Network (LIN) infrastructure, the infrastructure can utilize signals directly from automotive sensors 715. These sensors may detect internal light, temperature, touch or proximity sensors. External sensors can also be used to detection motion, interface distance measurements, touch sensing, video and/or other monitoring 717.

Further, the system 700 includes one or more control flash memory for storing data and various operational parameters during use. A network remote control 721 can be used to control the UV source 701 and diagnostics, behaviors and other data displayed on a display monitor 723. A control area network (CAN) bus 725 can be used to join and communicate with other systems wirelessly or directly through a radio transceiver and antenna system 727 using Bluetooth (BLE) or Mesh/Wi-Fi networks.

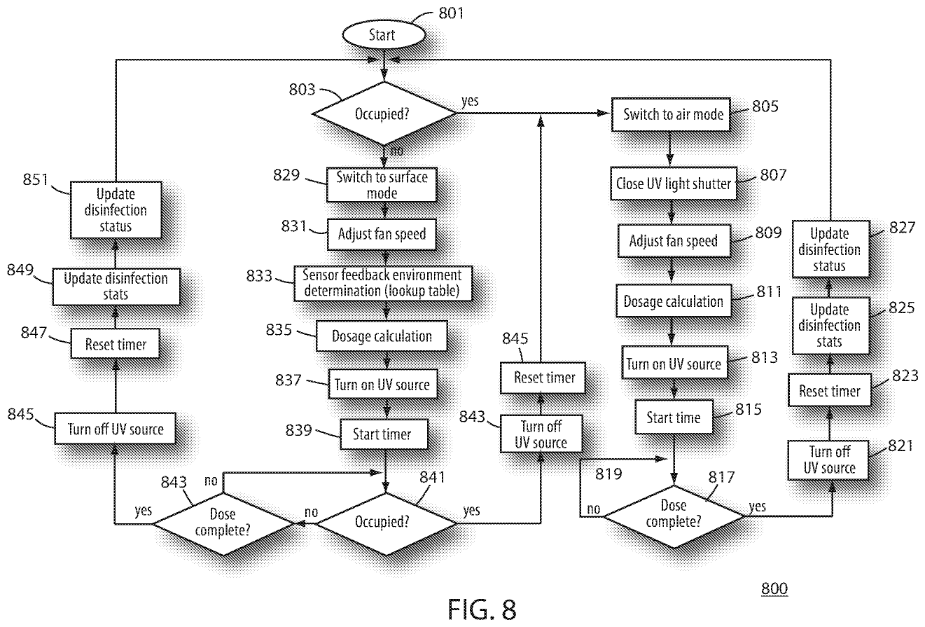

FIG. 8 is a flow chart diagram illustrating the disinfection processes used in the operation used in the dual mode disinfection system. The disinfection processes 800 starts 801 where a determination is made if the vehicle is occupied 803. If the vehicle is occupied, then the system is switched 805 solely to an air disinfection mode since exposure to UV-C rays within the vehicle cabin may be unwanted by human occupants. The UV light shutter is closed 807 and the fan motor speed within the device housing is adjusted 809.

Thereafter, a dosage calculation is made 811 and one or more UV-C light sources are activated 813. A start time is determined 815, and after some predetermined time, a determination is made if the UV-C dosage is complete 817. If not complete, the process continues 819. When complete, the UV-C light source is turned off 821 and the timer is reset 823. Disinfection data and statistics are updated 825 and vehicular disinfection status is updated 827. Thereafter, a determination is again made if the vehicle is occupied 803.

In the event the vehicle is not occupied, the system can be switched to surface disinfection mode 829. The fan speed internal to the device is adjusted 831 and a look-up table is used to determine UV-C dosage 833. The dosage is typically based on sensor feedback in the cabin environment. A dosage calculation is made 835 and one or more UV-C light sources are activated 837. A timer is started 839 and another determination is made 841 if the vehicle is occupied. If the vehicle is occupied by a driver or passengers, the UV source(s) are disabled 843 and the timer is reset 845. Thereafter, the process begins again to determine 803 if the vehicle is occupied.

Once the time 839 is started and the vehicle is not occupied 841, the UV-C light remains on until a determination is made if the dosage is complete 843. If not, complete, the UV-C sources remain in an on state, but if complete, the UV sources are turn to an off state and are disabled. The timer is reset 847 and any disinfection data and/or statistics are updated 849. The system status is thereafter updated 851 and the process can begin again if the vehicle is occupied 803.

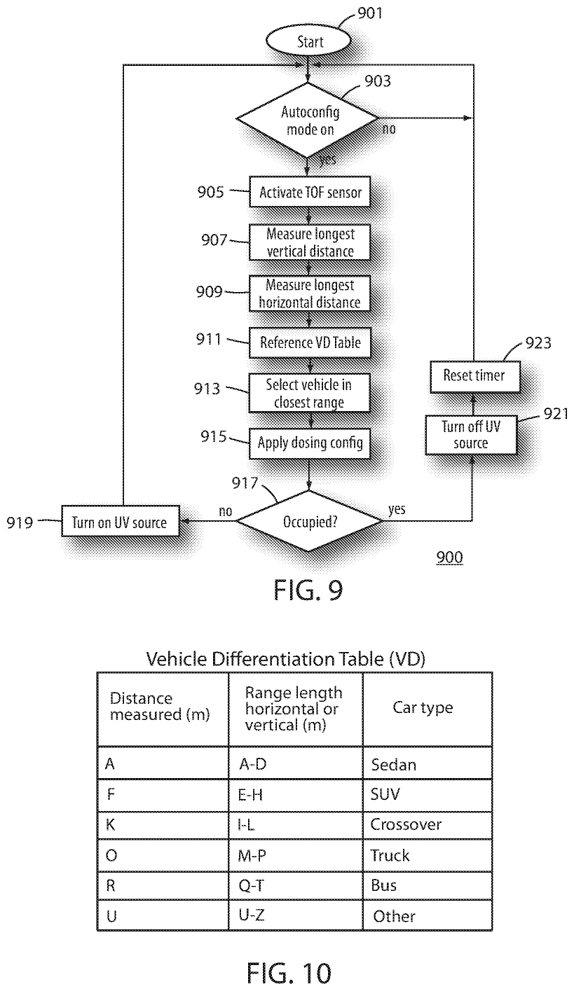

FIG. 9 is a flow chart diagram illustrating the autocalibration process 900 and methods for the dual mode disinfection system. The process starts 901 where the autocalibration process is initiated 901. In use, this may be a mode configured within a microprocessor or controller allowing the user to auto-calibrate the dual mode disinfection system without the need to measure interior surface distances from the UV-C light source. If the autoconfiguration mode is off, then the system will stand ready until actuated. Once the system is activated, a time-of-flight (ToF) or other type of distance measuring sensor is activated 905. As noted herein, a ToF sensor is a camera that measures distance by actively illuminating an object with a modulated light source such as a laser and a sensor that is sensitive to the laser's wavelength for capturing reflected light. The sensor measures the time delay difference between when the light is emitted and when the reflected light is received by the camera. The sensor operates by measuring, calculating and/or determining a longest/maximum vertical distance 907 and a longest/maximum horizontal distance 909. Once the maximum vertical distance and maximum horizontal distance are known, a vehicle differentiation (VD) table is used.

FIG. 10 is a chart or table illustrating vehicle differentiation (VD) used in the autocalibration process of FIG. 9. As seen in FIG. 10, once these distances are known, the distance is correlated to a range length. For example, range lengths A-D, E-H, I-L, M-P, Q-T, U-Z are each associated with a certain vehicle type. In this example, a sedan corresponds to a range length A-D. Once the autocalibration system knows the appropriate range length, it selects the vehicle having the correct range 913 and applies the proper UV-C light dosing configuration to the UV-C light sources.

A determination is then made if the vehicle is occupied 917. When occupied, the UV light sources are kept in an off or disabled state 921 and a timer is reset 923. The process then starts again 901 until it is determined that persons are no longer occupying the vehicle interior. When it is determined that no persons occupy the vehicle, the UV-C light source(s) are turned to an on or enabled state 919 for the proper dosing cycle, where after some predetermined time, the process will begin again 901. Hence, both FIGS. 9 and 10 illustrate an system where the UV-C disinfection process can be accurately calibrated without the needed for vehicle owners to preset UV-C lighting direction or dosages.

Hence, the present invention is directed to multiple embodiments of an HMI such as a control panel, keyboard or the like where a safe and useful dose of ultra-violet C (UV-C) light is applied to the surface of an HMI to provide disinfection of pathogens. Automating the combination of multiple devices with multiple intensities. The need to disinfect a control panel and HMI interfaces and spaces creates a both geometrical problem and a human interface problem. The geometrical problems are centered around available space and light coverage. the available space is often very limited and the coverage can be directed to the most often used areas of the touch screen that need disinfection.

Those skilled in the art, will recognize that the human interface issue relates to the cycle of contact to the touch screen. The occurrence of a human interacting with the interface at an event is counted as an event or “cycle” which will require disinfection when a task using the device is completed. High touch events like touch screen use, vehicular key and operational switches, vehicle control interfaces, and other devices that are frequency used are high touch devices. In order to disinfect the complete environment, there is a need to disinfect these high touch areas on a control panel/HMI interface, and then the general area between inter-human usage. Automating this process allows disinfection while in normal operating workflow and between users or events.

As described herein, a highly effective solution uses precision lighting delivery control with active and passive control of UV-C light having dynamically moveable multi-reflectors. A light distribution system is described herein having various filtering and reflective properties. Both are important as it is necessary to redistribute the UV-C energy effectively and safely around the product. The reflective properties can allow portions of the system to have different reflective properties allowing varied energy to move through and across the touch screen surface. The other benefit of these properties is the occurrence of a light scattering effect that assists in disinfection. Various embodiments of the invention use a multi-reflector configuration that simultaneously works together to change intensities and distribution of UV-C light in the system. This system of reflective surface can adjust intensity of the light to touchscreen usage rates, but also to programmed patterns for example, scanning vs. continuous vs. intermittent use. Disinfection requires providing an effective dose of UV-C radiation to the entire surface of the HMI. Many mounting scenarios preclude the mounting of a UV-C source orthogonally to the HMI's surface due to physical packaging reasons or aesthetic concerns and this solution offers a useful alternative.

The solutions provided herein offer control of intensity and distribution of UV-C light for both efficacy and safety. Those skilled in the art will further recognize that registering a series of events are important to automate a disinfection process. In one example, user movement can be tracked using infra-red (IR) sensors where the disinfection light distribution can be controlled by the preprogrammed movement of reflective surfaces. The reflective surfaces control the planar distribution of UV-C light to optimize disinfection as well as minimizing user exposure. This ensures the requirement to maintain the safety of an operator, or anyone in the vicinity of the HMI. The use of UV-C light can present a hazard, therefore excessive exposure must be avoided.

The present invention can also use remote LTV-C sensors for confirming a desired UV-C dose. By locating UV sensors in the farthest-reaching areas of the touch screen, the invention works to sense the UV-C intensity by adjusting a UV-C source to the required dose thus sparing materials in the environment additional UV-C exposure.

In another embodiment, light delivery can be adjusted to available multiple HMI screen surface bezel apertures. HMIs, control panels and touch screens, as used in practical applications have differing constraints on available bezel aperture dimensions. The design and ornamental requirements of the bezel can dictate particular solutions that can be used. As described herein, a manual or preprogrammed adjustable dynamic multi-mirror design helps deliver effective disinfection dose, to any size of surface, by the appropriate adjustment of a dynamic multi-reflector.

Analytics of the internal and external data captured can be used to provide insights into the necessary disinfection parameters. The invention also works to provide “value added” data to users by utilizing insights generated from both the analytic effects of sensors and treatment data. These might take the form of a graded dosage due to workload or seasonal patterns detected in data, that could help users of systems to improve efficiency or the reliance on materials that are compatible with UV-C.

Finally, further embodiments of the invention are directed to a system and method for providing a dual mode ultraviolet (UV) disinfection. The system includes but is not limited to a housing typically mounted to a vehicular headliner having a moveable shutter integrated therein. Those skilled in the art will recognize that the headliner is only one mounting option, and other mounting options e.g. the vehicles A-pillar are also possible. At least one light source is used within the housing for emitting light in the UV-C spectrum. A primary reflector partially surrounds the light source for directing light in a first predetermined direction. A secondary reflector can be configured forward of the light source for directing light towards the primary reflector. The primarily reflector and secondary reflector can direct the UV-C light to either disinfect air moving though the housing or direct light through the moveable shutter to designated locations within a vehicle interior. In still yet other embodiments, a shutter is optional where the UV-C lighting can be mounted on separate printed circuit (PC) boards allowing a first group of UV-C LEDs to be lit for air disinfection and a second groups of UV-C LEDs to be lit for surface disinfection.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A method for providing dual mode UV automotive disinfection comprising the steps of:
providing at least one UV light source;
moving automotive cabin air in proximity to the at least one UV light source;
configuring a shutter in proximity to the at least one UV light source in either an open or closed position;
controlling operation of the at least one UV light source, motor and shutter; and
operating the least one UV light source in a first mode by disinfecting cabin air in proximity to the at least one UV light source or in a second mode using the at least one light source to irradiate cabin surfaces.

2. A method for providing dual mode UV automotive disinfection as in claim 1, further comprising the steps of:
disinfecting cabin air when persons are detected in proximity to the as least one first sensor.

3. A method for providing dual mode UV automotive disinfection as in claim 1, further comprising the step of:
utilizing at least one sensor or sensor signal, for determining the presence of persons in the cabin and distances of cabin surfaces from the at least one UV light source.

4. A method for providing dual mode UV automotive disinfection as in claim 1, comprising the steps of:
automatically configuring a position and intensity of the at least one UV light source based on cabin surface location.

5. A method for providing dual mode UV automotive disinfection as in claim, further comprising the steps of:
providing at least one memory for storing position and intensity information of the at least one UV light source based upon vehicle make and model.

6. A method for providing dual mode UV automotive disinfection for the cleaning of automotive cabin air and interior surfaces comprising:
providing a housing mounted inside an automotive cabin;
configuring at least one UV light source inside the housing;
moving cabin air in proximity to the at least one UV light source using a motor;
moving a shutter configured in proximity to the at least one UV light between a first mode or second mode;
controlling operation of the at least one UV light source, motor and shutter; and
operating the shutter in a first mode so the at least one UV light source disinfects cabin air or in a second mode to disinfect cabin surfaces.

7. A method for providing dual mode UV automotive disinfection as in claim 6, further comprising the steps of:
closing the shutter for allowing cabin air to move through the housing when operated in the first mode.

8. A method for providing dual mode UV automotive disinfection as in claim 6, comprising the steps of:
opening the shutter for allowing light to escape from the housing when operated in the second mode.

9. A method for providing dual mode UV automotive disinfection as in claim 6, comprising the steps of:
mounting the housing to a headliner in an automotive cabin as one possible location with line of sight to surfaces of interest.

10. A method for providing dual mode UV automotive disinfection as in claim 6, further comprising the steps of:
providing at least one sensor or imported sensor signal, for determining the presence of persons within proximity to the at least one UV light source.

11. A method for providing dual mode UV automotive disinfection as in claim 10, further comprising the steps of:
disinfecting cabin air when persons are detected in proximity to the at least one sensor.

12. A method for providing dual mode UV automotive disinfection as in claim 6, further comprising the steps of:
providing at least one sensor for detecting a distance of cabin surfaces from the at least one UV light source.

13. A method for providing dual mode UV automotive disinfection as in claim 6, further comprising the step of:
automatically configuring a position and an intensity of the at least one UV light source based on cabin surface location.

14. A method for providing dual mode UV automotive disinfection as in claim 6, further comprising the step of:
providing at least one memory for storing position and intensity information of the UV light source based upon vehicle make and model.

15. A method of dual mode UV-C disinfection for the cleaning of automotive cabin air and surfaces comprising:
providing at least one UV light source;
moving cabin air in proximity to the at least one UV light source using a motor;
configuring a shutter in proximity to the at least one UV light source;
controlling operation of the at least one UV light source, motor and shutter using a controller;
determining the presence of persons within proximity to the at least one UV light source and detecting a distance of cabin surfaces from the at least one UV light source;
automatically configuring position and intensity of the at least one UV light source based on cabin surface location; and
operating the shutter in a first mode to disinfect cabin air or in a second mode to disinfect cabin surfaces depending on the position of the shutter.

16. A method of dual mode UV-C disinfection as in claim 15, further comprising the step of:
circulating cabin air through the housing when operated the shutter is closed and in the first mode.

17. A method of dual mode UV-C disinfection as in claim 16, further comprising the step of:
allowing UV light to escape from the housing to disinfect cabin surfaces when the shutter is open and in the second mode.

18. A method of dual mode UV-C disinfection as in claim 15, further comprising the steps of:
mounting the housing to a headliner of an automotive cabin.

19. A method of dual mode UV-C disinfection as in claim 15, further comprising the steps of:
storing position and intensity information of the UV light source based upon an automotive vehicle make and model in at least one memory.
